(19) United States
(12) Patent Application Publication
(10) Pub. No.: US 2010/0190742 A1
(43) Pub. Date: Jul. 29, 2010

(54) COSMETIC/PHARMACEUTICAL COMPOSITIONS COMPRISING HYALURONIC ACID AND ADMINISTRATION THEREOF FOR REDUCING SIGNS OF AGING

(75) Inventors: Lionel Breton, Versailles (FR); Jacques Leclaire, Massy (FR); Jean-François Grollier, Paris (FR)

Correspondence Address: BUCHANAN, INGERSOLL & ROONEY PC, POST OFFICE BOX 1404, ALEXANDRIA, VA, 22313-1404, US

(73) Assignee: L'OREAL, Paris (FR)

(21) Appl. No.: 12/704,978

(22) Filed: Feb. 12, 2010

Related U.S. Application Data
(63) Continuation of application No. PCT/FR2008/051473, filed on Aug. 7, 2008.

(30) Foreign Application Priority Data
Aug. 13, 2007 (FR) 0757054

(51) Int. Cl. A61K 31/715; A61P 17/00

(52) U.S. Cl. 514/54

(57) ABSTRACT

Cosmetic/pharmaceutical compositions contain, formulated into a physiologically acceptable medium, at least (i) hyaluronic acid and/or one of its derivatives and (ii) a C-glycoside compound, at least one of the constituents (i) and (ii) advantageously being in a form suitable for administration by injection, and administration thereof for filling depressions on the surface of the skin and for reducing signs of aging or for re-establishing the volume of the body or face.

### CROSS-REFERENCE TO PRIOR APPLICATIONS

This application claims priority under 35 U.S.C. §119 of FR 0757054, filed Aug. 13, 2007, and §120 of U.S. Provisional Application No. 60/935,777, filed Aug. 30, 2007, and is a continuation/national phase of PCT/FR 2008/051473, filed Aug. 7, 2008 and designating the United States (published in the French language on Feb. 26, 2009 as WO 2009/024719 A1; the title and abstract were also published in English), each hereby expressly incorporated by reference in its entirety and each assigned to the assignee hereof.

### BACKGROUND OF THE INVENTION

1. Technical Field of the Invention

The present invention relates to fillers which are particularly useful in the field of cosmetics and aesthetic dermatology. This invention relates to the formulation of such compositions having an improved effectiveness, comprising hyaluronic acid and derivatives thereof, and to their use.

2. Description of Background and/or Related and/or Prior Art

The human skin is composed of two compartments, namely, a surface compartment, the epidermis, and a deep compartment, the dermis.

The natural human epidermis is composed mainly of three types of cells which are keratinocytes, melanocytes and Langerhans' cells, the vast majority being keratinocytes. Each of these cell types contributes by its specific functions to the essential role played by the skin in the body.

The dermis provides the epidermis with a solid support. It is also its source of nutrients. It is mainly composed of fibroblasts and of an extracellular matrix itself composed mainly of collagen, of elastin and of a substance referred to as ground substance, which components are synthesized by the fibroblast. It also comprises leucocytes, mastocytes or tissue macrophages. It is also traversed by blood vessels and nerve fibers.

It is the collagen fibers which provide the dermis with strength. The collagen fibers are composed of fibrils firmly attached to one another, thus forming more than ten types of different structures. The strength of the dermis is largely due to the entanglement of the collagen fibers, which are packed tight against one another in all directions. The collagen fibers contribute to the elasticity and to the tonicity of the skin and/or mucous membranes.

The collagen fibers are constantly replaced but this replacement decreases with age, which results in a thinning of the dermis. In addition, various factors cause damage to the collagen, with all the consequences which can be envisaged with regard to the structure and/or the firmness of the skin and/or mucous membranes.

During the aging process, various characteristic signs appear on the skin, being reflected in particular by a change in the skin structure and functions. The main clinical signs of skin aging are in particular the appearance of fine lines and/or wrinkles, which increase with age. These wrinkles can be deep, moderate or superficial and they affect in particular the nasolabial folds, the periorbital region, the outline of the lips and the forehead (glabellar lines); these wrinkles and fine lines are reflected by a depression or folds at the surface of the skin.

Furthermore, a loss in volume of the upper part of the face, in particular at the cheekbones and cheeks, is observed.

Furthermore, there are various situations where it is desirable to correct the loss of tissues and to fill skin depressions, such as fibroblast depletions, wounds or the partial surgical removal of tissues (for example in the case of a tumor), including filling secondary skin “depressions” via a treatment resulting in lipodystrophy, which is generally characterized by a facial lipoatrophy, and to promote healing.

Various techniques have been provided for combating wrinkles and/or loss in volume of soft tissues.

In particular, one of such solutions is the use of filling products (or fillers). This filling can be carried out by the use of non-resorbable products, such as polyacrylamide gels or polymethyl methacrylate (PMMA) particles. Nevertheless, these compounds can bring about intolerance reactions of the inflammation or hypersensitivity type.

The use of resorbable components is favored, such as proteins, fats, collagen or hyaluronic acid. However, these compounds are decomposed quite rapidly in the body, which reduces their effectiveness. To overcome this, it is therefore necessary to carry out a more or less thorough crosslinking of these components, which is harmful to their tolerance and can make it more problematic, by modifying their physical state, to formulate them.

To date, the hyaluronic acid included in pharmaceutical forms or medical devices is provided in the form of a sodium hyaluronate gel. It is widely used by virtue of its ease of injection and its safety and it is a good alternative due to its biocompatibility and its absence of toxicity.

These sodium hyaluronate gels are furthermore widely employed in eye surgery. However, their rapid bioresorbability (typically varying from 4 to 6 months) can disappoint some users in the field of the filling of wrinkles or skin depressions, as injections have to be repeated at frequent and regular intervals.

There thus exists a need to develop fillers which are well tolerated and which exhibit a duration of action not requiring repeated applications.

### SUMMARY OF THE INVENTION

It has now been determined that the above and other objectives are achieved by conjoint administration of C-glycosides.

The present invention thus features cosmetic/pharmaceutical compositions, comprising, formulated into a physiologically acceptable medium, at least

(i) hyaluronic acid and/or derivatives thereof, and

(ii) a C-glycoside compound.

At least one of the constituents (i) or (ii) is in a form suitable for administration by injection or in the form of an injectable composition.

The composition provides an aesthetic and/or restoring purpose. An injectable compound or composition or a compound or composition suitable for administration by the injectable route is generally sterile.

The composition is advantageously suitable for administration which is simultaneous, separate or spread out over time.

Hyaluronic acid is formed by the repetition of a hydrophilic disaccharide unit, in which sodium D-glucuronate is connected to N-acetylglucosamine via β1-4 glycoside bonds. Its chemical structure is simple, linear and uniform, without species specificity.

In the physiological condition, the polysaccharide occurs not in the acid form but in the form of a sodium salt, sodium hyaluronate, which is a polyanionic molecule. By convention, the term “hyaluronan” is used. In healthy tissues, its molecular weight varies from 4 to 10 million daltons (400 D per unit), which gives a final product which can reach 10 μm. Only the length and the concentration of hyaluronan vary in nature. Hyaluronic acid is widely distributed in animal tissues and occurs at a high concentration in the synovial fluid, the vitreous humor of the eye, the connective tissues and the dermis. There are approximately 15 grams of hyaluronic acid in a human being weighing 70 kg, half of which is present in the skin. The dermis comprises from 0.5 to 0.74 mg thereof per g of wet tissue and the epidermis comprises from 0.10 to 0.15 mg thereof per g of wet tissue. The molecular weight of dermal hyaluronan is, on average, 2.5 million daltons. Its content varies as a function of age.

### DETAILED DESCRIPTION OF BEST MODE AND SPECIFIC/PREFERRED EMBODIMENTS OF THE INVENTION

Hyaluronic acid (HA) means, accordingly to the present invention, hyaluronic acid or of its salts, in particular its sodium, potassium, magnesium and calcium salts. Its molecular weight can vary and is generally greater than 10⁶ daltons. Within the meaning of the invention, hyaluronic acid is also understood to denote a mixture of molecules of different molecular weights, corresponding to more or less numerous repetitions of the base disaccharide unit.

The decomposition of hyaluronic acid takes place by virtue of the combined action of 3 different hyaluronidases. The relative contribution of the endo- and ex-cleavage reactions varies from one tissue to another. The half life of hyaluronic acid, due to the very rapid catabolism of the molecule, also varies from one tissue to another. By way of indication, it is approximately 1 day in the dermis and epidermis.

Industrially, hyaluronic acid can be obtained by extraction of tissues (for example of cockscombs) or by bacterial fermentation (such as Streptococcus equi).

Hyaluronic acid is soluble in water, its rate of dissolution being dependent on the molecular weight (the higher the molecular weight, the more slowly it will dissolve); the process can be accelerated by stirring.

Due to its short half life, the product should generally be subjected to crosslinking for use thereof in the context of the treatment of wrinkles by filling. Crosslinking takes place essentially in an alkaline medium, using the carboxyl and hydroxyl sites of the molecule. However, this crosslinking can be carried out in an acidic medium, although the bonds created in this medium are markedly weaker than in an alkaline medium. The crosslinking process does not change the polyanionic nature of the polysaccharide but substantially reduces the miscibility with water of the gel obtained.

In the crosslinking processes, the hyaluronic acid employed is of pharmaceutical quality. The conventional crosslinking technique comprises 3 stages: a stage of preparation of a hyaluronic acid of pharmaceutical quality, followed by drying, leaving the hyaluronan in the form of fibers. The tangle of hyaluronan fibers obtained is redissolved in a buffer solution. For example, hyaluronic acid of low molecular weight is mixed with an acid of high molecular weight. At this stage, the crosslinking agent is added, in alkaline medium, to give a gelled mass. The different crosslinking agents used to produce the gels available in cosmetic surgery are:

divinyl sulfone,

1,2,7,8-diepoxyoctane,

1,4-butanediol diglycidyl ether (BDDE), for a large number of products currently on the market;

in certain cases, a 4th stage, either by addition of hyaluronic acid or by a second stage of crosslinking in an acidic medium.

According to the present invention, hyaluronic acid derivatives means in particular chemically modified and/or crosslinked derivatives (exhibiting intra- or interchain bridges). Preferably, the crosslinked derivative is selected from among crosslinked derivatives with a molecular weight of greater than or equal to 10⁶ daltons and in particular of greater than or equal to 2 million daltons. However, this molecular weight is generally less than or equal to 15×10⁶ daltons.

It has now been discovered that a C-glycoside compound makes it possible to optimize the receptor binding of hyaluronic acid to its transmembrane receptor.

The present invention thus makes it possible to improve the bioavailability of hyaluronic acid or its derivatives, in particular when they are administered by the injectable route, and thus to reduce the frequency of the injections. To improve the bioavailability means in particular to reduce the rate of decomposition and/or to increase the concentration at the site of action.

The administration of compositions according to the invention thus makes it possible to improve the transcutaneous distribution of hyaluronic acid, or its crosslinked derivatives, in the skin by expression of the hyaluronic acid receptor, also known as CD44.

This invention also features administration of a C-glycoside derivative as an agent which induces expression of the receptor for hyaluronic acid or one of its derivatives.

Via the binding thereof with hyaluronic acid, and its internalization, CD44 participates in the replacement of the extracellular matrix and in cell mobility (by interaction with the cytoskeleton).

It is at the surface of T lymphocytes that a CD44 isoform was described for the first time. Various names have been assigned to this molecule, by reference sometimes to its function, sometimes to its structure, thus emphasizing the impact of CD44 in numerous physiological processes. CD44 is a family of transmembrane glycoproteins. The smallest isoform—without embodiment—is known as CD44 “standard” and is expressed in the majority of tissues. On the other hand, the expression of the isoforms comprising embodiments characterizes some tissues which are rapidly replaced, such as the skin. In the skin, the function of CD44 as hyaluronic acid receptor renders it crucial in the organization of the extracellular matrix.

CD44 also plays an important role in the phenomenon of healing. This is because the wounded tissues produce an enormous amount of hyaluronic acid, which forms a complex with the fibrin deposited by the blood. The fibroblasts will be bound, via their membrane CD44, to the hyaluronic acid, thus migrating into the injured tissue. This fibroblast will reconstruct the healthy tissue.

A C-glycoside compound suitable according to the present invention can be a compound of the following general formula (I):

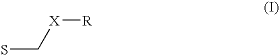

in which:

R is a saturated linear C₁ to C₂₀, in particular C₁ to C₁₀, alkyl radical, an unsaturated linear C₂ to C₂₀, in particular C₂ to O₁₀, alkyl radical or a saturated or unsaturated, branched or cyclic, C₃ to C₂₀, in particular C₃ to C₁₀, alkyl radical;

a saturated linear C₁ to C₂₀, in particular C₁ to C₁₀, hydrofluoroalkyl or perfluoroalkyl radical, an unsaturated linear C₂ to C₂₀, in particular C₂ to C₁₀, hydrofluoroalkyl or perfluoroalkyl radical or a saturated or unsaturated, branched or cyclic, C₃ to C₂₀, in particular C₃ to C₁₀, hydrofluoroalkyl or perfluoroalkyl radical;

with the proviso that the hydrocarbon chain constituting the said radicals can be, if appropriate, interrupted by 1, 2, 3 or more heteroatoms selected from among:

an oxygen,

a sulfur,

a nitrogen, and

a silicon,

and with the added proviso that the hydrocarbon chain constituting the said radicals may optionally be substituted by at least one radical selected from among:

—OR₄,

—SR₄,

—NR₄R₅,

—COOR₄,

—CONHR₄,

—CN,

a halogen atom,

a Cᵢ to C₆ hydrofluoroalkyl or perfluoroalkyl radical, and/or

a C₃ to C₈ cycloalkyl radical,

with the added proviso that R₄ and R₅ may be, independently of one another, a hydrogen atom, a saturated linear C₁ to C₃₀, in particular C₁ to C₁₂, alkyl, perfluoroalkyl or hydrofluoroalkyl radical, an unsaturated linear C₂ to C₃₀, in particular C₂ to C₁₂, alkyl, perfluoroalkyl or hydrofluoroalkyl radical, a saturated or unsaturated, branched or cyclic, C₃ to C₃₀, in particular C₃ to C₁₂, alkyl, perfluoroalkyl or hydrofluoroalkyl radical or a C₆ to C₁₀ aryl radical,

X is a radical selected from among the groups:

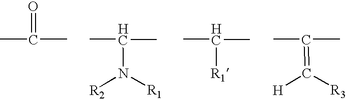

wherein R₁, R₂ and R₃ are each, independently of one another, a hydrogen atom or an R radical, wherein R is defined above, and R′₁ is a hydrogen atom, an —OH group or an R radical as defined above, with the proviso that R₁ may also be a C₆ to C₁₀ aryl radical,

S is a monosaccharide or a polysaccharide comprising up to 20 sugar units, in particular up to 6 sugar units, in a pyranose and/or furanose form and of the L and/or D series, with the proviso that the said mono- or polysaccharide may be substituted by a necessarily free hydroxyl group and optionally one or more optionally protected amine functional group(s), and

the S—CH₂—X bond is a bond of C-anomeric nature, which can be α or β,

and their cosmetically acceptable salts, their solvates, hydrates, and isomers thereof.

In the context of the present invention, the term “halogen” means chlorine, fluorine, bromine or iodine.

The term “aryl” is an aromatic ring, such as phenyl, optionally substituted by one or more C₁-C₄ alkyl radicals.

The term “C₃ to C₈ cycloalkyl” is an aliphatic ring member having from 3 to 8 carbon atoms, including, for example, cyclopropyl, cyclopentyl and cyclohexyl.

Exemplary alkyl radicals include the methyl, ethyl, isopropyl, n-propyl, n-butyl, t-butyl, isobutyl, sec-butyl, pentyl, n-hexyl, cyclopropyl, cyclopentyl, cyclohexyl and allyl radicals.

According to one embodiment of the invention, a C-glycoside compound is employed corresponding to the formula (I) in which S is a monosaccharide or polysaccharide comprising up to 6 sugar units, in the pyranose and/or furanose form and of the L and/or D series, the said mono- or polysaccharide exhibiting at least one necessarily free hydroxyl functional group and/or optionally one or more necessarily protected amine functional groups, X and R furthermore retaining all of the definitions given above.

Advantageously, a monosaccharide according to the invention can be selected from among D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine or N-acetyl-D-galactosamine and advantageously is D-glucose, D-xylose, N-acetyl-D-glucosamine or L-fucose, in particular D-xylose.

More particularly, a polysaccharide according to the invention comprising up to 6 sugar units can be selected from among D-maltose, D-lactose, D-cellobiose, D-maltotriose, a disaccharide combining a uronic acid selected from among D-iduronic acid or D-glucuronic acid with a hexosamine selected from among D-galactosamine, D-glucosamine, N-acetyl-D-galactosamine or N-acetyl-D-glucosamine, or an oligosaccharide comprising at least one xylose which can advantageously be selected from among xylobiose, methyl p-xylobioside, xylotriose, xylotetraose, xylopentaose and xylohexaose and in particular xylobiose, which is composed of two xylose molecules connected via a 1,4-bond.

More particularly, each S can be a monosaccharide selected from among D-glucose, D-xylose, L-fucose, D-galactose or D-maltose and in particular D-xylose.

According to another embodiment of the invention, a C-glycoside compound is employed corresponding to the formula (I) in which X is a group selected from among —CO—, —CH(OH)—, —CH(NR₁R₂)— or —CH(R)—, in particular —CO—, —CH(OH)—, —CH(NH₂)—, —CH(NHCH₂CH₂CH₂OH)—, —CH(NHPh)- or —CH(CH₃)—, and more particularly a CO—, —CH(OH)— or —CH(NH₂)— group and in particular a CH(OH)— group, S and R furthermore retaining all of the definitions given above.

According to another embodiment of the invention, a C-glycoside compound is employed corresponding to the formula (I) in which R is a saturated linear C₁ to C₂₀, in particular C₁ to C₁₀, alkyl radical, an unsaturated linear C₂ to C₂₀, in particular C₂ to C₁₀, alkyl radical or a saturated or unsaturated, branched or cyclic, C₃ to C₂₀, in particular C₃ to C₁₀, alkyl radical optionally substituted as described above, S and R furthermore retaining all of the definitions given above. Preferably, R is a linear C₁-C₄, in particular C₁-C₃, radical optionally substituted by OH, —COOH or —COOR″₂, R″₂ being a saturated C₁-C₄ alkyl radical, in particular an ethyl radical. Preferably, R is an unsubstituted linear C₁-C₄, in particular C₁-C₂, alkyl radical, especially ethyl.

Particularly preferred are the C-glycoside compounds of formula (I), in which:

R is a saturated linear C₁ to C₂₀, in particular C₁ to C₁₀, alkyl radical, an unsaturated linear C₂ to C₂₀, in particular C₂ to C₁₀, alkyl radical or a saturated or unsaturated, branched or cyclic, C₃ to C₂₀, in particular C₃ to C₁₀, alkyl radical optionally substituted as described above;

S is a monosaccharide as described above;

X is —CO—, —CH(OH)—, —CH(NR₁R₂)— or —CH(R)— as described above.

In particular, a C-glycoside compound of formula (I) is employed, in which:

R is a linear C₁-C₄, in particular C₁-C₃, radical optionally substituted by —OH, —COON or —COOR″₂, R″₂ being a saturated C₁-C₄ alkyl radical, in particular ethyl;

S is a monosaccharide as described above;

X is a group selected from among —CO—, —CH(OH)—, —CH(NH₂)—, —CH(NHCH₂CH₂CH₂OH)—, —CH(NHPh)- or —CH(CH₃)—, more particularly a —CO—, —CH(OH)— or —CH(NH₂)— group and preferably a —CH(OH)— group.

More particularly, a C-glycoside compound of formula (I) is employed, in which:

R is an unsubstituted linear C₁-C₄, in particular C₁-C₂, alkyl radical, in particular ethyl;

S is a monosaccharide as described above, in particular D-glucose, D-xylose, N-acetyl-D-glucosamine or L-fucose and especially D-xylose;

X is a group selected from among —CO—, —CH(OH)— or —CH(NH₂)—, and preferably a —CH(OH)— group.

The salts acceptable for non-therapeutic administration of the compounds described in the present invention comprise conventional nontoxic salts of the said compounds, such as those formed from organic or inorganic acids.

Exemplary salts of inorganic acids are those of sulfuric acid, hydrochloric acid, hydrobromic acid, hydriodic acid, phosphoric acid or boric acid.

Exemplary salts of organic acids are those which can comprise one or more carboxylic, sulfonic or phosphonic acid groups. They can be linear, branched or cyclic aliphatic acids or also aromatic acids. These acids can additionally comprise one or more heteroatoms selected from among O and N, for example in the form of hydroxyl groups. Exemplary are those of propionic acid, acetic acid, terephthalic acid, citric acid and tartaric acid.

When the compound of formula (I) comprises an acid group, the acid group or groups can be neutralized with an inorganic base, such as LiON, NaOH, KOH, Ca(OH)₂, NH₄OH, Mg(OH)₂ or Zn(OH)₂; or with an organic base, such as a primary, secondary or tertiary alkylamine, for example triethylamine or butylamine. This primary, secondary or tertiary alkylamine can comprise one or more nitrogen and/or oxygen atoms and can thus comprise, for example, one or more alcohol functional groups; exemplary are those of 2-amino-2-methylpropanol, triethanolamine, 2-(dimethylamino)propanol or 2-amino-2-(hydroxymethyl)-1,3-propanediol. Also exemplary are those of lysine or 3-(dimethylamino)propylamine.

The solvates acceptable for the compounds according to the present invention comprise conventional solvates, such as those formed during the final stage of preparation of the said compounds as a result of the presence of solvents. Exemplary are the solvates due to the presence of water or of linear or branched alcohols, such as ethanol or isopropanol.

Very particularly preferred C-glycoside compounds of formula (I) according to the invention are:
1. C-β-D-xylopyranoside-n-propan-2-one;
2. C-α-D-xylopyranoside-n-propan-2-one;
3. 1-[2-(3-hydroxypropylamino)propyl]-C-β-D-xylopyranose;
4. 1-[2-(3-hydroxypropylamino)propyl]-C-α-D-xylopyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-phenylaminopropane;
10. C-α-D-xylopyranoside-2-phenylaminopropane;
11. ethyl ester of 3-methyl-4-(C-α-D-xylopyranoside)butyric acid;
12. ethyl ester of 3-methyl-4-(C-β-D-xylopyranoside)butyric acid;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-phenylaminohexanoic acid;
20. 6-(C-α-D-xylopyranoside)-5-phenylaminohexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-phenylaminopentanoic acid;
30. 5-(C-α-D-xylopyranoside)-4-phenylaminopentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-phenylaminopropane;
46. 1-(C-α-D-fucopyranoside)-2-phenylaminopropane;
47. 1-(C-β-L-fucopyranoside)-2-phenylaminopropane;
48. 1-(C-α-L-fucopyranoside)-2-phenylaminopropane;
49. ethyl ester of 3-methyl-4-(C-β-D-fucopyranoside)butyric acid;
50. ethyl ester of 3-methyl-4-(C-α-D-fucopyranoside)butyric acid;
51. ethyl ester of 3-methyl-4-(C-β-L-fucopyranoside)butyric acid;
52. ethyl ester of 3-methyl-4-(C-α-L-fucopyranoside)butyric acid;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)hexane-2,6-diol)-4-ketopentanoic acid;
72. 5-(C-α-L-fucopyranoside)hexane-2,6-diol)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-fucopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-phenylaminopropane;
90. 1-(C-α-D-glucopyranosyl)-2-phenylaminopropane;
91. ethyl ester of 3-methyl-4-(C-β-D-glucopyranosyl)butyric acid;
92. ethyl ester of 3-methyl-4-(C-α-D-g lucopyranosyl)butyric acid;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-phenylaminohexanoic acid;
100. 6-(C-α-D-glucopyranosyl)-5-phenylamino-hexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-hydroxypentanoic acid;
109. 6-(C-β-D-glucopyranosyl)-5-phenylaminopentanoic acid;
110. 6-(C-α-D-glucopyranosyl)-5-phenylaminopentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-phenylaminopropane;
118. 1-(C-α-D-galactopyranosyl)-2-phenylaminopropane;
119. ethyl ester of 3-methyl-4-(β-D-galactopyranosyl)butyric acid;
120. ethyl ester of 3-methyl-4-(α-D-galactopyranosyl)butyric acid;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-phenylaminohexanoic acid;
128. 6-(C-α-D-galactopyranosyl)-5-phenylaminohexanoic acid;
129. 1-(C-β-D-galactopyranosyl)hexane-2,6-diol;
130. 1-(C-α-D-galactopyranosyl)hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-phenylaminopentanoic acid;
138. 6-(C-α-D-galactopyranosyl)-5-phenylaminopentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propan-2′-one;
146. 3′-(acetamido-C-α-D-glucopyranosyl)propan-2′-one;
147. 1-(acetamido-C-β-D-glucopyranosyl)-2-hydroxypropane;
148. 1-(acetamido-C-β-D-glucopyranosyl)-2-aminopropane;
149. 1-(acetamido-C-β-D-glucopyranosyl)-2-phenylaminopropane;
150. 1-(acetamido-C-α-D-glucopyranosyl)-2-phenylaminopropane;
151. ethyl ester of 3-methyl-4-(acetamido-C-β-D-glucopyranosyl)butyric acid;
152. ethyl ester of 3-methyl-4-(acetamido-C-α-D-glucopyranosyl)butyric acid;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-phenylaminohexanoic acid;
160. 6-(acetamido-C-α-D-glucopyranosyl)-5-phenylaminohexanoic acid;
161. 1-(acetamido-C-β-D-glucopyranosyl)hexane-2,6-diol;
162. 1-(acetamido-C-α-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-phenylaminopentanoic acid;
170. 6-(acetamido-C-α-D-glucopyranosyl)-5-phenylaminopentanoic acid;
171. 1-(acetamido-C-β-D-glucopyranosyl)pentane-2,5-diol;
172. 1-(acetamido-C-α-D-glucopyranosyl)pentane-2,5-diol.

More particularly preferred C-glycoside compounds according to the invention are:
C-3-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxypropane,
1-(C-α-D-glucopyranosyl)-2-hydroxypropane,
1-(C-β-D-galactopyranosyl)-2-hydroxypropane,
1-(C-α-D-galactopyranosyl)-2-hydroxypropane,
1-(C-β-D-fucofuranosyl)propan-2-one,
1-(C-α-D-fucofuranosyl)propan-2-one,
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one,
C-β-D-maltopyranoside-2-hydroxypropane,
C-α-D-maltopyranoside-2-hydroxypropane, their isomers and mixtures thereof.

According to one embodiment, C-β-D-xylopyranoside-2-hydroxypropane or C-α-D-xylopyranoside-2-hydroxypropane, and more particularly C-β-D-xylopyranoside-2-hydroxypropane, are advantageously employed for the formulation of a composition according to the invention.

Of course, according to the invention, a C-glycoside compound corresponding to the formula (I) can be used alone or as a mixture with other C-glycoside compounds and in all proportions.

According to one specific embodiment, the C-glycoside compound is C-β-D-xylopyranoside-2-hydroxypropane in the form of a 30% by weight solution of active material in a water/propylene glycol (60/40% by weight) mixture, such as the product marketed by Chimex under the trademark “Mexoryl SBB®”.

A C-glycoside compound suitable for the invention can in particular be obtained by the method of synthesis described in WO 02/051828.

The amount of C-glycoside compound to be employed in a composition according to the invention depends on the cosmetic, dermatological or therapeutic effect desired and can thus vary within wide limits.

One skilled in the art can easily, on the basis of his general knowledge, determine the appropriate amounts.

A composition according to the invention can comprise a C-glycoside compound in a proportion from approximately 0.0001 to approximately 25% by weight, with respect to the total weight of the composition, in particular from approximately 0.001 to approximately 10% by weight and more particularly from approximately 0.05 to 5% by weight of C-glycoside derivative active material, with respect to the total weight of the composition.

Likewise, the concentration of hyaluronic acid and/or its derivatives in the compositions according to the invention will be adjusted by one skilled in the art according to the molecular weight of the compounds and the effect desired. By way of indication, they can vary from 0.0001 to 25% by weight, in particular from 0.001 to 10% by weight. Concentrations from approximately 1 to 3% can in particular be employed.

A composition in accordance with the invention comprises a physiologically acceptable medium.

This physiologically acceptable medium can comprise a hydrophilic phase, in particular an aqueous phase, as or not as a mixture with one or more organic solvents, such as a C₁-C₈ alcohol, in particular ethanol, isopropanol, tert-butanol or n-butanol, polyols, such as glycerol, propylene glycol or butylene glycol, and polyol ethers.

Advantageously, the medium is an aqueous hydrophilic medium. The composition is preferably provided in the form of an aqueous dispersion comprising hyaluronic acid or one of its crosslinked derivatives.

Another aspect of the invention is a composition comprising at least (i) a first component comprising hyaluronic acid or one of its derivatives and at least (ii) a second component comprising a C-glycoside compound of the formula (I) as defined above, as combination product for administration which is simultaneous, separate or spread out over time in the treatment of losses in volume of the dermis and/or epidermis.

The compositions according to the invention will in particular be cosmetic or pharmaceutical compositions, in particular a dermatological composition.

According to one of its embodiments, it can be provided in a form suitable for application by the topical route, in particular the external topical route.

According to another embodiment of the invention, the composition can be in a form suitable for administration by the injectable route.

According to a specific embodiment, the composition comprises at least one component suitable for administration by the injectable route and at least one component suitable for administration by the external topical route useful for application on the skin or its appendages.

Preferably, hyaluronic acid or its derivatives are in the composition in a form suitable for administration by the injectable route. According to another embodiment, the C-glycoside compounds are present in a composition applied by the external topical route.

According to another embodiment of the invention, the C-glycoside compounds are present in a composition suitable for the injectable route.

The invention relates in particular to an injectable implant, in particular a dermal implant, based on hyaluronic acid in combination with a C-glycoside, by the subcutaneous or intradermal route. It is thus a dermal implant, that is to say substances injected directly into the skin for the purpose of curing the disorders occurring in the different cutaneous or subcutaneous levels (dermis, hypodermis, and the like) described above.

The compositions according to the invention are useful in particular for humans or animals in plastic surgery or in aesthetic dermatology for filling wrinkles, fine lines, skin depressions and scars, including filling secondary skin “depressions” in a treatment resulting in lipodystrophy, which is generally characterized by a facial lipoatrophy. The composition can be an implant as defined above.

According to one embodiment, the component or the composition suitable for administration by the injectable route is in the form of a gel or implant.

This is because these compositions can be administered subcutaneously or intradermally. This is a matter in particular of an injectable implant or a medical device which comprises at least one C-glycoside compound and at least HA or one of its derivatives or one of its analogues.

The molecular weight of the hyaluronic acid present in the compositions according to the invention can vary to a large extent, in particular from 10,000 to 8,000,000 daltons, in particular from 50,000 to 3 million daltons. In a preferred embodiment of the invention, the implant is composed of hyaluronic acid with a molecular weight of greater than one million daltons, preferably from one million to five million daltons.

The compositions according to the invention can also comprise formulation adjuvants known to one skilled in the art, such as, for example, stabilizing agents, preservatives or pH adjusters.

The compositions can in particular be provided in the form of aqueous, aqueous/alcoholic or oily solutions, of dispersions of a lotion or serum type, of anhydrous or oily gels, of emulsions with a liquid or semiliquid consistency of the milk type, obtained by dispersion of a fatty phase in an aqueous phase (O/W) or vice versa (W/O), of suspensions or emulsions with a soft, semi-solid or solid consistency of the cream or gel type, of microemulsions or also of microcapsules, of microparticles or of vesicular dispersions of ionic and/or nonionic type. These compositions are prepared according to the standard methods.

In the case of injectable compositions, the latter will comprise, for example, from 10 to 40 mg/ml of hyaluronic acid and/or its derivatives, in particular from 15 to 25 mg/ml; their pH is in particular from 7 to 7.5. The injectable compositions are preferably sterile compositions.

They will be formulated so as to maintain the sterility and to exclude the presence of endotoxins and/or pyrogenic agents. The osmolarity will also be controlled to be compatible with the administration route.

The physiologically acceptable medium can thus be essentially composed of water or of an isotonic aqueous solution. In addition, it can comprise buffers, viscosifiers and/or antioxidants.

According to one of the alternative embodiments of the invention, the composition or injectable implant additionally comprises another filler, in particular collagen or its crosslinked derivatives, as described, for example, in WO 01/79342, elastin and/or polylactic acid.

The composition can optionally comprise other anti-aging active principles, for example ascorbic acid or its derivatives.

The present invention also features a cosmetic treatment regime or regimen for reducing signs of aging of the skin and/or mucous membranes, in particular for reducing wrinkles and/or fine lines and/or restoring the volume thereof to the face or body, or for increasing the volume of the lips, in which a composition comprising a combination (i) of at least hyaluronic acid and/or one of its derivatives and (ii) of at least one C-glycoside compound as defined above is applied. The cosmetic treatment method according to the invention makes it possible in particular to erase or soften unsightly marks of scarring, for example resulting from acne; it also makes it possible to restore the outline of the body, for example in the context of breast implants, or the outline of the face, modified during aging.

In particular, the regime or regimen according to the invention comprises the injection of the said composition into at least one cutaneous or subcutaneous tissue.

The present invention also features a cosmetic treatment regime or regimen for reducing signs of aging of the skin or mucous membranes and/or for re-establishing the volume of the body or face, wherein a composition comprising hyaluronic acid or one of its derivatives as defined above is injected into at least one cutaneous or subcutaneous tissue. The C-glycoside compound will, in one embodiment, also be injected into a cutaneous or subcutaneous tissue; in another embodiment, the C-glycoside derivative will be applied in external topical fashion.

The regime or regimen according to the invention preferably comprises an injection at a single site or at a limited number of points of the cutaneous or subcutaneous tissues. It can be repeated over time, in particular at six-monthly time intervals.

Another aspect of the invention is the formulation, into a composition, in particular a cosmetic or pharmaceutical composition, of at least one C-glycoside compound for improving the bioavailability and/or reducing the decomposition of hyaluronic acid or its crosslinked derivatives; advantageously, hyaluronic acid or its derivatives are present in the said composition.

The present invention also features administration of a C-glycoside compound as an agent which induces expression of the hyaluronic acid receptor.

To further illustrate the present invention and the advantages thereof, the following specific examples are given, it being understood that same are intended only as illustrative and in nowise limitative. In said examples to follow, all parts and percentages are given by weight, unless otherwise indicated.

### Example

### Effect of C-β-D-xylopyranoside-2-hydroxypropane on the expression of CD44

Principle of the Test:

CD44 is a transmembrane glycoprotein with a molecular weight of 80-95 kDa (CD44). The increase in the expression of this receptor at the surface of the cells of the skin improves the binding of hyaluronic acid, collagen and fibronectin. This binding makes possible in particular hydration of the dermis and epidermis.

This receptor can be demonstrated, on skin explants treated daily with C-β-D-xylopyranoside-2-hydroxypropane (compound A), by a S-layer indirect immunoperoxidase technique.

Protocol:

Normal human skin fragments are obtained by plastic surgery and are deposited in culture plates. The skin explants are cultured for 14 days and compound A is added daily at 0.3 or 3 mM. At the end of culturing, the skin explants are fixed in Bouin's fluid and embedded in paraffin wax. Sections are produced and the receptor is visualized by a 3-layer indirect immunoperoxidase technique (ABC peroxidase kit, Vector Laboratories).

Histological Evaluation:

This study shows a significant increase in the expression of CD44 with compound A.

This activity is present at 0.3 mM and is again encountered at a higher concentration (3 mM).

Expression of CD44:

Histochemical analysis of the CD44 receptor (semiquantitative scores);

| Treatment | Mean ± sd n = 8 |
|---|---|
| Control skin | 0.90 ± 0.90 |
| Skin + compound A at 0.3 mM | 2.30 ± 1.00* |
| Skin + compound A at 3.0 mM | 2.00 ± 1.10* |

*Statistically significant difference with respect to the control skin (p < 0.05)

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A cosmetic/pharmaceutical composition suited for durable aesthetic/restorative dermatology applications, comprising (i) an enhancedly bioavailable amount of hyaluronic acid and/or derivative(s) thereof, and (ii) an amount of at least one C-glycoside compound which induces expression of the receptor for hyaluronic acid and/or derivative(s) thereof, formulated into (iii) a physiologically acceptable medium therefor.

2. The cosmetic/pharmaceutical composition as defined by claim 1, at least one of said constituents (i) and/or (ii) being formulated for administration by injection.

3. The cosmetic/pharmaceutical composition as defined by claim 1, comprising at least one hyaluronic acid derivative selected from among those crosslinked derivatives having a molecular weight of greater than or equal to 10⁶ daltons.

4. The cosmetic/pharmaceutical composition as defined by claim 1, said at least one C-glycoside compound having the following formula (I): 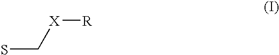
in which:
R is a saturated linear C₁ to C₂₀ alkyl radical, an unsaturated linear C₂ to C₂₀ alkenyl radical or a saturated or unsaturated, branched or cyclic, C₃ to C₂₀ hydrocarbyl radical;
a saturated linear C₁ to C₂₀ hydrofluoroalkyl or perfluoroalkyl radical, an unsaturated linear C₂ to C₂₀ hydrofluoroalkenyl or perfluoroalkenyl radical or a saturated or unsaturated, branched or cyclic, C₃ to C₂₀ hydrofluorocarbyl or perfluorhydrocarbyl radical;
with the proviso that the hydrocarbon chain constituting the said radicals may optionally be interrupted by 1, 2, 3 or more heteroatoms selected from among:
an oxygen,
a sulfur,
a nitrogen, and
a silicon atom;
and with the further proviso that the hydrocarbon chain constituting the said radicals may optionally be substituted by at least one radical selected from among:
—OR₄,
—SR₄,
—NR₄R₅,
—COOR₄,
—CONHR₄,
—CN,
a halogen atom,
a C₁ to C₆ hydrofluoroalkyl or perfluoroalkyl radical, and/or
a C₃ to C₈ cycloalkyl radical;
with the further proviso that R₄ and R₅ may independently be a hydrogen atom, a saturated linear C₁ to C₃₀ alkyl, perfluoroalkyl or hydrofluoroalkyl radical, an unsaturated linear C₂ to C₃₀ alkenyl, perfluoroalkenyl or hydrofluoroalkenyl radical, a saturated or unsaturated, branched or cyclic, C₃ to C₃₀ hydrocarbyl, perfluorohydrocarbyl or hydrofluorocarbyl radical or a C₆ to C₁₀ aryl radical;
X is a radical selected from among the groups: 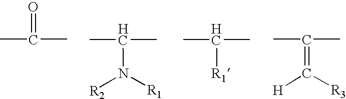
wherein R₁, R₂ and R₃, independently, are each a hydrogen atom or an R radical, wherein R is as defined above, and R′₁ is a hydrogen atom, an —OH group or an R radical as defined above, with the proviso that R₁ may also be a C₆ to C₁₀ aryl radical;
S is a monosaccharide or a polysaccharide comprising up to 20 sugar units, in a pyranose and/or furanose form and of the L and/or D series, with the proviso that the said mono- or polysaccharide may optionally be substituted by a necessarily free hydroxyl group and optionally one or more optionally protected amine functional group(s); and
the S—CH₂—X bond is a C-anomeric bond, which can be α or β, and the cosmetically acceptable salts, solvates, hydrates, and isomers thereof.

5. The cosmetic/pharmaceutical composition as defined by claim 1, wherein S is a monosaccharide selected from among D-glucose, D-xylose, L-fucose, D-galactose and D-maltose.

6. The cosmetic/pharmaceutical composition as defined by claim 1, wherein X is a group selected from among —CO—, —CH(OH)— and —CH(NH₂)—.

7. The cosmetic/pharmaceutical composition as defined by claim 4, in which R is a linear C₁-C₄ radical optionally substituted by —OH, —COON or —COOR″₂, wherein R″₂ is a saturated C₁-C₄ alkyl radical, in particular an ethyl radical.

8. The cosmetic/pharmaceutical composition as defined by claim 1, in which the at least one C-glycoside compound is selected from among:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxypropane,
1-(C-α-D-glucopyranosyl)-2-hydroxypropane,
1-(C-β-D-galactopyranosyl)-2-hydroxypropane,
1-(C-α-D-galactopyranosyl)-2-hydroxypropane,
1-(C-β-D-fucofuranosyl)propan-2-one,
1-(C-α-D-fucofuranosyl)propan-2-one,
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one,
C-β-D-maltopyranoside-2-hydroxypropane,
C-α-D-maltopyranoside-2-hydroxypropane, and the isomers and mixtures thereof.

9. The cosmetic/pharmaceutical composition as defined by claim 1, in which the at least one C-glycoside compound is selected from C-β-D-xylopyranoside-2-hydroxypropane and C-α-D-xylopyranoside-2-hydroxypropane.

10. The cosmetic/pharmaceutical composition as defined by claim 1, comprising the at least one C-glycoside compound in a proportion from approximately 0.00001% to approximately 15% by weight, with respect to the total weight of the composition.

11. The cosmetic/pharmaceutical composition comprising at least (i) a first component comprising hyaluronic acid and/or one of its derivatives and at least (ii) a second component comprising at least one C-glycoside compound of the formula (I) as defined in claim 1, formulated for simultaneous, separate or administration spread out over time.

12. The cosmetic/pharmaceutical composition as defined by claim 1, formulated for administration by the injectable route.

13. The cosmetic/pharmaceutical composition as defined by claim 1, comprising at least one component suitable for administration by the injectable route and at least one component suitable for administration by the topical route.

14. The cosmetic/pharmaceutical composition as defined by claim 1, at least one component thereof being suitable for administration by the injectable route and formulated as a gel or an implant.

15. The cosmetic/pharmaceutical composition as defined by claim 1, further comprising at least one other filler material.

16. A regime or regimen for filling depressions on the surface of the skin, comprising treating such surface with a thus effective amount of a cosmetic/pharmaceutical composition as defined by claim 1.

17. The regime or regimen as defined by claim 16, comprising treating wrinkles and fine lines and/or restoring volume to the face.

18. The regime or regimen as defined by claim 16, wherein the at least one C-glycoside comprises C-β-D-xylopyranoside-2-hydroxypropane and/or derivative thereof.

19. A regime or regimen for reducing signs of aging of the skin or mucous membranes and/or for re-establishing the volume of the body or face, comprising injecting a cosmetic/pharmaceutical composition as defined by claim 1 into at least one affected cutaneous or subcutaneous tissue.

20. A regime or regimen for the treatment of losses in volume of the dermis and/or epidermis in the skin of an individual in need of such treatment, comprising administering thereto a thus effective amount of the cosmetic/pharmaceutical composition as defined by claim 1.

21. A regime or regimen for reducing signs of aging of the skin and/or mucous membranes, for reducing wrinkles and/or fine lines and/or restoring the volume thereof to the face or body, or for increasing the volume of the lips, comprising administering to an individual in need of such treatment, a thus effective amount of the cosmetic/pharmaceutical composition as defined by claim 1.

22. A regime or regimen for reducing signs of aging of the skin and/or mucous membranes, for reducing wrinkles and/or fine lines and/or restoring the volume thereof to the face or body, or for increasing the volume of the lips, comprising injecting into the affected cutaneous and/or subcutaneous tissue of an individual in need of such treatment, (i) an enhancedly bioavailable amount of hyaluronic acid and/or derivative(s) thereof, or (ii) an amount of at least one C-glycoside compound which induces expression of the receptor for hyaluronic acid and/or derivatives thereof, and topically applying thereon an effective amount of the other of said constituents (i) and (ii) not injected.

24. A regime or regimen for the treatment of losses in volume of the dermis and/or epidermis in the skin of an individual in need of such treatment, comprising separately administering thereto a thus effective amount of each of the constituents (i) and (ii) of the cosmetic/pharmaceutical composition as defined by claim 1.
